(19) United States
(12) Patent Application Publication
(10) Pub. No.: US 2003/0055225 A1
(43) Pub. Date: Mar. 20, 2003

(54) Monoclonal antibody for the detection of protein products of the brx gene, and uses thereof

(76) Inventors: Domenica Marie Rubino, Falls Church, VA (US); Paul Driggers, Falls Church, VA (US)

Correspondence Address: Robert M. Schulman, Esq., Hunton & Williams, Suite 1200, 1900 K Street, N.W., Washington, DC, 20006, US

(21) Appl. No.: 09/953,407

(22) Filed: Sep. 17, 2001

(51) Int. Cl. A61K 39/395; C12P 21/04; C07K 16/40

(52) U.S. Cl. 530/388.26; 435/70.21; 424/146.1

(57) ABSTRACT

A monoclonal antibody specific for the brx gene product, Brx, implicated in the etiology of breast and ovarian cancers is disclosed. Further disclosed are methods of using this antibody to diagnose and treat proliferative disorders of reproductive and immune tissues including cancer. Methods of use of the monoclonal antibody as a molecular biological reagent for applications including but not limited to Western analysis, immunohistochemistry, immunoprecipitation, gel shift analysis, enzyme-linked immunosorption assays are also disclosed.

### FIELD OF THE INVENTION

This invention pertains to the fields of oncology and reproductive biology. It generally relates to oncogenes, particularly their use to diagnose disease and in therapeutic regimens. In particular, this invention pertains to the development of a monoclonal antibody for the detection of products of the gene brx, implicated in the etiology of breast and ovarian cancers. The invention relates to methods of using the monoclonal antibody to diagnose, detect, and treat certain cancers and diseases and disorders of reproductive and immune tissues. The invention also relates to methods of use of the monoclonal antibody as a molecular biological reagent for applications including but not limited to Western analysis, immunohistochemistry, immunoprecipitation, gel shift analysis, enzyme-linked immunosorption assays.

### BACKGROUND OF THE INVENTION

One in eight women will develop breast cancer. There are 200,000 new cases per year. In addition, 26,000 cases of ovarian cancer are diagnosed each year. Ovarian cancer has the highest mortality rate of gynecological cancer.

Some cancerous conditions may be arrested, if diagnosed and treated in a timely fashion, and the life of the patient thereby prolonged. However, in many cases, the cancer is discovered after it has advanced to the point that the fatal progress of the disease cannot be reversed, and the patient's life cannot be saved. Thus, it is important to develop methods for the early detection, diagnosis, and treatment of cancer.

Additionally, proliferative disorders in reproductive tissues such as endometriosis affect many women and abnormal response of such tissues to hormone regulation may result in implantation abnormalities and/or luteal insufficiency.

Some existing methods of cancer diagnosis and treatment are targeted to tumor-specific proteins. Proteins that are specifically associated with certain tumors have been described as early as 1965. Garrett and Kurtz, Medical Clinics of North America 70:1295-1306 (1986) defined the ideal biological tumor marker as one that is a) specific without false-positive results, b) sensitive without false-negative results and c) capable of demonstrating an absolute correlation with the extent of disease. In particular, tumor-associated proteins or tumor markers would be a powerful tool in detecting malignancies if they were present only in tumor cells. However, although most of the currently identified tumor markers are present in high yields in tumor cells, they are also found in non-tumor cells.

Other methods of diagnosis and treatment are targeted to tumor-active nucleic acids. Many cancers are believed to result from a series of genetic alterations leading to progressive disordering of normal cellular growth mechanisms (Nowell, Science 194:23 (1976); Foulds, J. Chronic Dis. 8:2 (1958)). The deletion or multiplication of copies of whole chromosomes or chromosomal segments, or specific regions of the genome are common (see, e.g., Smith, et al., (1991) Breast Cancer Res. Treat., 18: Suppl. 1: 5-14; van de Vijer & Nusse (1991) Biochim. Biophys. Acta. 1072: 33-50; Sato, et al., (1990) Cancer. Res., 50: 7184-7189). In particular, the amplification and deletion of DNA sequences containing proto-oncogenes and tumor-suppressor genes, respectively, are frequently characteristic of tumorigenesis. Dutrillaux, et al., Cancer Genet. Cytogenet., 49: 203-217 (1990). Substances that modulate the action or effect of oncogenes are likely therapeutic agents.

Auxiliary or co-activator proteins have been described which regulate the transcriptional effects of members of the nuclear hormone receptor (NHR) superfamily (T3R, VDR, RAR, PPAR, RXR, ER). These proteins reportedly govern tissue-specific hormone responses of NHRs. Activation of the estrogen receptor (ER) is reportedly affected by ligand-dependent phosphorylation, and co-activator proteins as in the tripartite model of receptor activation (reviewed in Katzenellenbogen et al., Mol. Endocrinol. 10: 129-129 (1996)). Prior to Rubino, et al, Rho proteins had not been shown to be directly involved in ER activation. Rho proteins act as binary molecular switches through incompletely defined interactions with specific target effector proteins to direct transcription, cell proliferation, cell adhesion, actin cytoskeletal organization, protein kinase signaling (Watanabe et al., Science 271:645-648 (1996)) and oncogenesis (Bos, Cancer Res. 49: 4682-4689 (1989)). Substantial evidence suggests that signals generated by p21 GTPases (especially p21^{ras}) are transmitted to the nucleus. Hill et al., Cell 81: 1159-1170 (1995); Minden et al., Cell 81: 1147-1157 (1995); Settleman et al., Cell 69: 539-549 (1992); Hunter & Karin, Cell 70: 375-387 (1992); Hall, Annu. Rev. Cell Biol 10: 1-54 (1994). Involvement of Rho in nuclear hormone receptor signaling was suggested by the description of p190, a rho/rac GAP, which exists in both nuclear and cytoplasmic compartments, transduces signals from p21^{ras} to the nucleus (Settleman et al., Cell 69: 539-549 (1992)), and may suppress glucocorticoid receptor transcription (LeClerc et al., J. Biol. Chem. 266: 17333-17340 (1991)). Brx is a novel member of the Dbl (Rho-GEF) family that is capable of augmenting ligand-dependent estrogen receptor function in reproductive tissues (Rubino, et al., 1998).

Furthermore, the estrogen and progesterone receptor status of breast tumors has been shown to be of prognostic importance, but there are few tests for ovarian cancer, thus necessitating the identification of new prognostic markers for this condition. The present invention addresses this and other current problems.

### SUMMARY OF THE INVENTION

One embodiment of the present invention provides a method of detection of novel proteins, which constitute growth regulatory proteins or oncoproteins useful in the regulation of cell proliferation in cell cultures and in vivo, particularly proteins active in reproductive epithelium, immune cells, endometrium, testes, ovary, breast cells, spleen, and brain.

Another embodiment of the present invention provides a method of detection of novel proteins, useful for stimulating or suppressing tumor cell growth, particularly in reproductive epithelium, immune cells, endometrium, testes, ovary, breast, spleen and brain.

Another embodiment of the present invention provides a novel monoclonal antibody, useful for detection of diagnostic and/or prognostic markers in proliferative and other disorders of reproductive and immune tissues and brain.

Another embodiment of the present invention provides a novel monoclonal antibody, useful for detection of diagnostic and/or prognostic markers in the diagnosis and/or therapy of patients in need thereof.

Another embodiment of the present invention provides a novel monoclonal antibody, useful for pharmaceutical tests of cancer and immune function.

Another embodiment of the present invention provides a novel monoclonal antibody, useful as a molecular biological reagent for applications including but not limited to Western analysis, immunohistochemistry, immunoprecipitation, gel shift analysis, enzyme-linked immunosorption assays.

Another embodiment of the present invention provides generally safe and specific therapeutic and prophylactic methods and products useful for controlling growth disorders, including cancer in reproductive and immune tissues.

A further embodiment of the present invention provides products and methods of controlling cancer that are specific for eradication of the cancer tumor by utilizing biotechnological methods and products.

According to another embodiment the present invention provides for cells (e.g., recombinant cells such as hybridomas or triomas) expressing the above-described monoclonal antibody.

According to a further embodiment, the present invention provides for kits for the detection and/or quantification of a brx gene product. The kit can include a container containing the above identified antibody with or without labels, free, or bound to a solid support as described herein. The kits can also include instructions for the use of one or more of these reagents in any of the assays described herein.

According to a still further embodiment, the present invention provides for methods of detecting a predisposition to breast or ovarian cancer, proliferative, and other disorders of reproductive, immune, and brain tissues. The methods include the steps of i) providing a biological sample of the organism; and ii) detecting the presence, absence and/or level of expression of brx gene protein product in the sample. The provision of a biological sample and detection methods are known in the art or described herein. In particular, detecting can involve detecting the presence or absence, or quantifying a brx gene protein product. The detecting can also involve detecting the presence or absence or quantifying a Brx protein sequence or subsequence thereof. The detecting can involve detecting the presence or absence of normal or abnormal Brx polypeptides.

According to an even further implementation, the present invention also provides therapeutic methods. These include methods of diagnosing, preventing or treating cancers, proliferative, and other diseases of reproductive epithelium, immune cells, ovary, breast, endometrium, testis, spleen and brain, particularly in a mammal such as a human patient. Other methods involve administering to the mammal a therapeutically effective dose of a composition comprising a Brx monoclonal antibody and a pharmacological excipient as described herein. The methods are preferably performed on mammals such as mice, rats, rabbits, sheep, goats, pigs, more preferably on primates including human patients.

A further embodiment of the present invention provides a monoclonal antibody comprising:

(i) a polypeptide that specifically binds to a ΔN3-Brx protein at about the C-terminal region, said C-terminal region comprising SEQ ID NO:2;

(ii) a polypeptide that specifically binds to a full length Brx protein (SEQ ID NO:1) at about the C-terminal region, said C-terminal region comprising SEQ ID NO:2; or

(iii) a polypeptide that specifically binds at about the C-terminal region of any Brx protein derived from the same gene as the ΔN3-Brx protein, said Brx protein being larger than the ΔN3-Brx protein and including but not limited to a protein comprising SEQ ID NO:1; or

(iv) a polypeptide that specifically binds to a protein comprising SEQ ID NO. 2.

A further embodiment of the present invention provides a monoclonal antibody prepared by a process comprising:

introducing into an animal a ΔN3-Brx protein comprising SEQ ID No:2 and

recovering a monoclonal antibody from the animal.

A further embodiment of the present invention provides a polynucleotide comprising:

(i) a nucleic acid sequence that encodes a polypeptide that specifically binds to a ΔN3-Brx protein at about the C-terminal region, said C-terminal region comprising SEQ ID NO:2;

(ii) a nucleic acid sequence that encodes a polypeptide that specifically binds to a full length Brx protein (SEQ ID NO:1) at about the C-terminal region, said C-terminal region comprising SEQ ID NO:2;

(iii) a nucleic acid sequence that encodes a polypeptide that specifically binds at about the C-terminal region of any Brx protein derived from the same gene as the ΔN3-Brx protein, said Brx protein being larger than the ΔN3-Brx protein and including but not limited to a protein comprising SEQ ID NO:1; or

(iv) a nucleic acid sequence that encodes a polypeptide that specifically binds to a protein comprising SEQ ID NO. 2.

A further embodiment of the present invention provides a transformed cell line comprising:

(i) a recombinant cell expressing a polypeptide that specifically binds to a ΔN3-Brx protein at about the C-terminal region, said C-terminal region comprising SEQ ID NO:2;

(ii) a recombinant cell expressing a polypeptide that specifically binds to a full length Brx protein (SEQ ID NO:1) at about the C-terminal region, said C-terminal region comprising SEQ ID NO:2; or

(iii) a recombinant cell expressing a polypeptide that specifically binds at about the C-terminal region of any Brx protein derived from the same gene as the ΔN3-Brx protein, said Brx protein(s) being larger than the ΔN3-Brx protein and including but not limited to a protein comprising SEQ ID NO:1; or

(iv) a recombinant cell expressing a polypeptide that specifically binds to a protein comprising SEQ ID NO. 2.

A further embodiment of the present invention provides a composition comprising:

(a) a polypeptide or plurality of polypeptides selected from the group consisting of (i) polypeptides that specifically bind to a ΔN3-Brx protein at about the C-terminal region, said C-terminal region comprising SEQ ID NO:2; (ii) polypeptides that specifically bind to a full length Brx protein (SEQ ID NO:1) at about the C-terminal region, said C-terminal region comprising SEQ ID NO:2; (iii) polypeptides that specifically bind at about the C-terminal region of a Brx protein derived from the same gene as the ΔN3-Brx protein, said Brx protein being larger than the ΔN3-Brx protein and including but not limited to a protein comprising SEQ ID NO:1; (iv) polypeptides that specifically binds to a protein comprising SEQ ID NO. 2, and (v) combinations thereof; and

(b) a pharmaceutically acceptable carrier.

A further embodiment of the present invention provides a kit comprising:

(a) a polypeptide or plurality of polypeptides selected from the group consisting of (i) polypeptides that specifically bind to a ΔN3-Brx protein at about the C-terminal region, said C-terminal region comprising SEQ ID NO:2, (ii) polypeptides that specifically bind to a full length Brx protein at about the C-terminal region, said C-terminal region comprising SEQ ID NO:2; (iii) polypeptides that specifically bind at about the C-terminal region of a Brx protein derived from the same gene as the ΔN3-Brx protein, said Brx protein being larger than the ΔN3-Brx protein and including but not limited to a protein comprising SEQ ID NO:1, (iv) polypeptides that specifically binds to a protein comprising SEQ ID NO. 2, and (v) combinations thereof; and

(b) a reagent for detecting a monoclonal antibody-ΔN3-Brx protein complex in a patient or in a sample of tissue from the patient.

A further embodiment of the present invention provides a method for detecting ΔN3Brx protein in a patient comprising:

introducing to the tissue of a patient a composition comprising a polypeptide or plurality of polypeptides selected from the group consisting of (i) polypeptides that specifically bind to a ΔN3-Brx protein at about the C-terminal region, said C-terminal region comprising SEQ ID NO:2, (ii) polypeptides that specifically bind to a full length Brx protein (SEQ ID NO:1) at about the C-terminal region, said C-terminal region comprising SEQ ID NO:2, (iii) polypeptides that specifically bind at about the C-terminal region of a Brx protein derived from the same gene as the ΔN3-Brx protein, said Brx protein being larger than the ΔN3-Brx protein and including but not limited to a protein comprising SEQ ID NO:1, (iv) polypeptides that specifically binds to a protein comprising SEQ ID NO. 2, and (v) a combination thereof; and

detecting the presence, absence and/or level of a brx gene protein product in the tissue.

A further embodiment of the present invention provides a method of protecting a patient against infection and/or colonization by ΔN3-Brx protein comprising:

administering to the patient an effective amount of a composition comprising a polypeptide or plurality of polypeptides selected from the group consisting of (i) polypeptides that specifically bind to a ΔN3-Brx protein at about the C-terminal region, said C-terminal region comprising SEQ ID NO:2; (ii) polypeptides that specifically bind to a full length Brx protein (SEQ ID NO:1) at about the C-terminal region, said C-terminal region comprising SEQ ID NO:2; (iii) polypeptides that specifically bind at about the C-terminal region of a Brx protein derived from the same gene as the ΔN3-Brx protein, said Brx protein being larger than the ΔN3-Brx protein and including but not limited to a protein comprising SEQ ID NO:1, (iv) a polypeptide that specifically binds to a protein comprising SEQ ID NO. 2, and (v) combinations thereof.

A further embodiment of the present invention provides a method of producing a monoclonal antibody comprising:

introducing into the cells of an animal an immunogen comprising the polypeptide of SEQ ID NO:2; and

recovering the monoclonal antibody from the cells of the animal.

### DESCRIPTION OF PREFERRED EMBODIMENTS

A. Definitions

Unless defined otherwise, all technical and scientific terms used herein have the same meaning as commonly understood by one of ordinary skill in the art to which this invention belongs.

A cell line is said to be “malignant” if, when the cell line is injected into a host animal, the host animal develops tumors or cancers that are anaplastic, invasive, and/or metastatic. A “human” tumor is comprised of cells that have human chromosomes. Such tumors include those in a human patient, and tumors resulting from the introduction of a human malignant cell line into a non-human host animal if cells from such tumors have human chromosomes.

The terms “treating cancer”, “therapy”, and the like mean generally a treatment that causes any improvement in a mammal having a cancer wherein the improvement can be ascribed to treatment with a Brx monoclonal antibody. The improvement can be either subjective or objective. For example, if the mammal is human, the patient may note improved vigor or vitality or decreased pain as subjective symptoms of improvement or response to therapy. Alternatively, the clinician may notice a decrease in tumor size or tumor burden based on physical exam, laboratory parameters, tumor markers, or radiographic findings.

The term “effective amount” means a dosage sufficient to produce a desired result. The desired result can be subjective or objective improvement in the recipient of the dosage, a decrease in tumor size, a decrease in the rate of growth of cancer cells, or a decrease in metastasis.

“Inhibiting the growth of cancer cells” is evaluated by any accepted method of measuring whether growth of the cancer cells has been slowed or diminished. This includes direct observation and indirect evaluation such as subjective symptoms or objective signs as discussed below.

“Subsequence” refers to a sequence of amino acids that comprise a part of a longer sequence of amino acids (e.g., polypeptide) respectively.

The phrase “specifically binds to an antibody” or “specifically immunoreactive with”, when referring to a protein or peptide, refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to an antibody under such conditions may require an antibody that is selected for its specificity for a particular protein. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988) ANTIBODIES, A LABORATORY MANUAL, Cold Spring Harbor Publications, New York, for a description of immunoassay formats and conditions that can be used to determine specific immunoreactivity.

B. Detailed Description

(1) Brx Monoclonal Antibody

The present invention provides for a monoclonal antibody specific to Brx. Preferably, the monoclonal antibody is mouse or rabbit monoclonal antibody. More preferably, the monoclonal antibody is mouse monoclonal antibody. The particular antibody specifically binds a protein comprising the C-terminal domain, and larger Brx protein(s), including but not limited to the protein sequence given in Rubino et al. 1998 and GENBANK accession number AF126008. The C-terminal region possesses some of the biological properties of the published protein, Brx: e.g., the ability to specifically bind to GST-RXRβ, ERα/β and other NHRs, the ability to modestly enhance activation by the estrogen receptor, as well as bind to transcription factors known to be involved in mitogenesis: c-Jun, c-Fos and ATF-2.

The present invention is based, at least in part, on the unexpected discovery of novel receptor-binding proteins in breast cell lines, without intending to be limited by theory. A breast cancer cDNA expression library (ZR75-1; Clontech) was screened using a modified method of interaction cloning; a recombinant, epitope-tagged, bacterially expressed nuclear receptor (RXR); and commercially available antibodies directed against the epitope (FLAG-IBI). The screening yielded several clones, one of which (clone 2.10) contained a 1.8 kB fragment. Two other cDNA expression libraries (human testis and breast cancer, Clontech) were probed with labeled clone 2.10 (ΔN3-brx, (amino acids 960-1428 of Brx), Rubino, et al.; SEQ ID NO:2) fragment, and multiple overlapping clones were isolated and sequenced (Rubino, et. al.). These cDNA fragments corresponded to a 5.3 kB mRNA transcript encoding a predicted 1428 amino acid protein (SEQ ID NO:1) with a 168 kilodalton molecular mass. The open reading frame is preceded by four “in-frame” stop codons and contains an initiator methionine surrounded by a Kozak consensus sequence. The protein was designated Brx, and the coding sequence was designated brx. brx cDNA sequence was found to contain a region of sequence identity to lbc, a partial cDNA associated with acute blast crisis of chronic myelogenous leukemia (Toksoz and Williams, reference included in its entirety). Analysis of Brx amino acid sequence (amino acids 1-1428) revealed that Brx contained a domain (amino acids 534-950) of identity to Lbc (Rubino, et al.). This evidence suggests that the brx gene can be mutated to a smaller oncogenic form, termed lbc and implies that alterations in Brx protein expression may be associated with malignancy. A description of the Brx protein was published in Rubino, et al, Oncogene, 1998, 16: 2513-2526, the disclosure of which is expressly incorporated herein by reference in its entirety.

The brx sequence has been mapped by FISH to chromosome 15q25-26. Some regions of chromosome 15q have been associated with breast cancer (Miller, et al.). Northern analysis showed that brx message was expressed in ovary, breast, breast cancer cell lines, testis, spleen, immune cells, thyroid and brain, but not in liver and pancreas.

Labeled Brx bound specifically to GST-RXRβ, ERα, and other NHRs including TR, RAR and PPAR. Brx also bound to ERβ (Rubino and Driggers). Brx bound to nuclear hormone receptors, including the estrogen receptor, through a novel carboxyl region. Furthermore, we observed that Brx augmented ligand-dependent gene activation by the estrogen receptors α (Rubino, et al) and β (Rubino and Driggers, submitted) and was localized to nuclear and cytoplasmic cellular compartments.

Specificity of binding of Brx to the estrogen receptor was confirmed in co-immunoprecipitation studies using an affinity matrix constructed with anti-estrogen receptor antibody and human recombinant ER. Transient transfection studies in Ishikawa endometrial cells revealed that Brx augments ligand-dependent ERα reporter activity. Thus, Brx enhances activation by the estrogen receptor. Experiments showed that enhancement of ER activation by Brx is dependent upon the small GTPase Cdc42Hs. Collectively, these results suggest a convergence of signaling pathways involving small GTPase proteins and nuclear hormone receptors.

Additionally, Brx binds to transcription factors known to be involved in mitogenesis: c-Jun, c-Fos, ATF-2 and modifies the serum response element, a DNA regulatory element regulating genes involved in cell proliferation. Brx was found to bind to the Rho GTPase family members, RhoA and Cdc42Hs in vitro. Furthermore, we expect that Brx will bind to isoforms of TR, PPAR, RAR, ER and RXR as well as other nuclear hormone receptors: androgen receptor (AR), progesterone receptor (PR), glucocorticoid receptor (GR) and their different isoforms.

According to an implementation, the present invention provides a mouse monoclonal antibody specific to Brx. The particular antibody specifically binds a protein comprising SEQ ID NO:2 at the C-terminal domain (amino acids 960-1428) of a Brx protein (SEQ ID NO:1; amino acid 1-1428). SEQ ID NO:1 is given in Rubino et al. 1998 and GENBANK accession number AF126008.

(2) Detection of brx Gene Products

Brx or a peptide fragment thereof may be detected or quantified by a variety of methods. Preferred methods involve the use of specific antibodies.

(A). Detection of Brx Proteins by Immunoassay

Methods of producing polyclonal and monoclonal antibodies are known to those of skill in the art. See, e.g., Coligan (1991), CURRENT PROTOCOLS IN IMMUNOLOGY, Wiley/Greene, NY; and Harlow and Lane (1989), ANTIBODIES: A LABORATORY MANUAL, Cold Spring Harbor Press, NY; Stites et al. (eds.) BASIC AND CLINICAL IMMUNOLOGY (4th ed.) Lange Medical Publications, Los Altos, Calif., and references cited therein; Goding (1986), MONOCLONAL ANTIBODIES: PRINCIPLES AND PRACTICE (2d ed.) Academic Press, New York, N.Y.; and Kohler and Milstein (1975), Nature, 256:495-497. Such techniques include antibody preparation by selection of antibodies from libraries of recombinant antibodies in phage or similar vectors. See, Huse et al. (1989), Science, 246:1275-1281; and Ward et al. (1989) Nature, 341:544-546. For example, in order to produce antisera for use in an immunoassay, the polypeptide of SEQ ID NO: 2 is isolated as described herein. For example, recombinant protein is produced in a transformed cell line. An inbred strain of mice or rabbits is immunized with the protein of SEQ ID NO: 2, using a standard adjuvant, such as Freund's adjuvant, and a standard immunization protocol. Alternatively, a synthetic peptide derived from the sequences disclosed herein and conjugated to a carrier protein can be used an immunogen. Polyclonal sera are collected and titered against the immunogen protein in an immunoassay, for example, a solid phase immunoassay with the immunogen immobilized on a solid support. Polyclonal antisera with a titer of 10⁴ or greater are selected and tested for their cross reactivity against non-Brx or even Brx from other cell types or species or a peptide fragment thereof, using a competitive binding immunoassay. Specific monoclonal and polyclonal antibodies and antisera will usually bind with a K_{D} of at least about 0.1 mM, more usually at least about 1 μM, preferably at least about 0.1 μM or better, and most preferably, 0.01 μM or better.

i. Antibody Production

A number of immunogens may be used to produce antibodies specifically reactive with Brx or a peptide fragment thereof. Recombinant protein is the preferred immunogen for the production of monoclonal antibodies. Naturally occurring protein may also be used either in pure or impure form. Synthetic peptides made using the Brx or a peptide fragment thereof sequences described herein may also used as an immunogen for the production of antibodies to the protein. Recombinant protein can be expressed in eukaryotic or prokaryotic cells as described above, and purified as generally described above. The product is then injected into an animal capable of producing antibodies. Monoclonal antibodies may be generated, for subsequent use in immunoassays to measure the protein.

Monoclonal antibodies may be obtained by various techniques familiar to those skilled in the art. Briefly, spleen cells from an animal immunized with a desired antigen are immortalized, commonly by fusion with a myeloma cell (See, Kohler and Milstein, Eur. J. Immunol. 6:511-519 (1976), incorporated herein by reference). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al. (1989) Science 246:1275-1281.

ii. Immunoassays

A particular protein can be measured by a variety of immunoassay methods. For a review of immunological and immunoassay procedures in general, see BASIC AND CLINICAL IMMUNOLOGY, 7th Edition (D. Stites and A. Terr, eds.) 1991. Moreover, the immunoassays of the present invention can be performed in any of several configurations, which are reviewed extensively in ENZYME IMMUNOASSAY, E. T. Maggio, ed., CRC Press, Boca Raton, Fla. (1980); “Practice and Theory of Enzyme Immunoassays,” P. Tijssen, LABORATORY TECHNIQUES IN BIOCHEMISTRY AND MOLECULAR BIOLOGY, Elsevier Science Publishers B. V. Amsterdam (1985); and, Harlow and Lane, ANTIBODIES, A LABORATORY MANUAL, supra, each of which is incorporated herein by reference.

Immunoassays to Brx or a peptide fragment thereof of the present invention may use a monoclonal antiserum, which was raised to the protein of SEQ ID NO:2. This antiserum is selected to have low crossreactivity against other (non-Brx or Brx) proteins and any such crossreactivity is removed by immunoabsorption prior to use in the immunoassay.

Immunoassays in the competitive binding format can be used for the crossreactivity determinations. For example, the protein of SEQ ID NO:2 can be immobilized to a solid support. Proteins (Brx-like, or non-Brx, or unknown) are added to the assay that compete with the binding of the antisera to the immobilized antigen. The ability of the above proteins to compete with the binding anti-BRX antisera or antibodies to the immobilized protein is compared to the protein of SEQ ID NO:2. The percent crossreactivity for the above proteins is calculated, using standard calculations. Those antisera with less than 10% crossreactivity with each of the proteins listed above are selected and pooled. Any cross-reacting antibodies are optionally removed from the pooled antisera by immunoabsorption with the above-listed proteins.

The immunoabsorbed and pooled antisera are then used in a competitive binding immunoassay as described herein to compare the binding of a second protein to that of the immunogen protein, ΔN3-Brx (SEQ ID NO:2). In order to make this comparison, the two proteins are each assayed at a wide range of concentrations and the amount of each protein required to inhibit 50% of the binding of the antisera to the immobilized protein is determined. If the amount of the second protein required is less than 10 times the amount of the protein of SEQ ID NO: 2 that is required, then the second protein is said to specifically bind to an antibody generated to an immunogen consisting of the protein of SEQ ID NO:2.

The presence of a desired polypeptide (including peptide, transcript, or enzymatic digestion product) in a sample may be detected and quantified using Western blot analysis. The technique generally comprises separating sample products by gel electrophoresis on the basis of molecular weight, transferring the separated proteins to a suitable solid support, (such as a nitrocellulose filter, a nylon filter, or derivatized nylon filter), and incubating the sample with labeling antibodies that specifically bind to the analyte protein. The labeling antibodies specifically bind to analyte on the solid support. These antibodies are directly labeled, or alternatively are subsequently detected using labeling agents such as antibodies (e.g., labeled sheep anti-mouse antibodies where the antibody to an analyte is a murine antibody) that specifically bind to the labeling antibody.

3. Pharmaceutical Compositions

The compositions for administration will commonly comprise a solution of the Brx antibody or antibody chimera/fusion dissolved in a pharmaceutically acceptable carrier, preferably an aqueous carrier. A variety of aqueous carriers can be used, e.g., buffered saline and the like, without limitation. These solutions are sterile and generally free of undesirable matter. These compositions may be sterilized by conventional, well-known sterilization techniques.

In certain embodiments, the Brx monoclonal antibody, are provided in powder form, or in a pharmaceutically acceptable solution such as an aqueous solution, often a saline solution, without limitation. The antibody may be combined with conventional excipients, such as pharmaceutical grades of mannitol, lactose, starch, magnesium stearate, sodium saccharin, talcum, cellulose, glucose, sucrose, magnesium, carbonate, and the like, or combinations thereof, for example, without limitation. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like, or combinations thereof, without limitation. The concentration of chimeric molecule in these formulations can vary widely, and will be selected primarily based on fluid volumes, viscosities, body weight and the like in accordance with the particular mode of administration selected and the patient's needs. The resulting compositions may be in the form of a solution, suspension, tablets, pill, capsule, powder, gel, cream, lotion, ointment, aerosol and the like, or combinations thereof, without limitation.

The pharmaceutical composition or medium that comprises Brx antibody is administered orally, parenterally, enterically, gastrically, topically, subcutaneously, locally, systemically or in combinations thereof, without limitation. It is recognized that the Brx antibody described above, when administered orally, must be protected from digestion. This is typically accomplished either by complexing the protein with a composition to render it resistant to acidic and enzymatic hydrolysis or by packaging the protein in an appropriately resistant carrier such as a liposome. Means of protecting proteins from digestion are well known in the art.

Thus, a typical pharmaceutical composition for intravenous administration would be about 0.1 to 10 mg per patient per day. Dosages from 0.1 up to about 1000 mg per patient per day may be used, particularly when the drug is administered to a secluded site and not into the blood stream, such as into a body cavity or into a lumen of an organ. Substantially higher dosages are possible in topical administration. Actual methods for preparing parenterally administrable compositions will be known or apparent to those skilled in the art and are described in more detail in such publications as Remington's Pharmaceutical Science, 15th ed., Mack Publishing Company, Easton, Pa. (1980). Preferably, the dosage is about 0.1 to about 1000 mg per patient per twenty-four hour period of time, more preferably about 0.1 to about 100 mg per patient per twenty-four hour period of time, and even more preferably about 0.1 to about 10 mg per patient per twenty-four hour period of time.

The compositions containing the present Brx antibodies or antibody chimer/fusions, or a cocktail thereof (i.e., with other proteins), can be administered for therapeutic treatments. In therapeutic applications, compositions are administered to a patient suffering from a disease (e.g., breast or basal cell carcinoma) in an amount sufficient to cure or at least partially arrest the disease and its complications. An amount adequate to accomplish this is defined as a “therapeutically effective dose.” Amounts effective for this use will depend upon the severity of the disease and the general state of the patient's health.

Single or multiple administrations of the compositions may be administered depending on the dosage and frequency as required and tolerated by the patient. In any event, the composition should provide a sufficient quantity of the proteins of this invention to effectively treat the patient.

4. Diagnostic Applications

Only a few proteins are known to bind to the estrogen receptor, and Brx appears to be one of these. The evidence presented below leads to the conclusion that brx may be a tumor suppressor gene, and that alterations in the expression pattern of the Brx protein may be associated with malignancy.

As discussed in Rubino, et al., brx cDNA sequence was found to contain a region of sequence identity to lbc, a partial cDNA associated with acute blast crisis of chronic myelogenous leukemia (Toksoz and Williams, reference included in its entirety). Analysis of Brx amino acid sequence (amino acids 1-1428) revealed that Brx contained a domain (amino acids 534-950) of 97% identity to Lbc (Rubino, et al.). This evidence suggests that the brx gene can be mutated to a smaller oncogenic form, termed lbc and implies that alterations in Brx protein expression may be associated with malignancy. Furthermore, analysis of brx messenger RNA reveals different sized messages in normal vs. malignant tissues, such as breast and immune tissue (Rubino, unpublished).

Thus, detection of Brx expression in cancer tissue sections (such as in cancers of the breast, ovaries, germinal epithelium, endometrium, testis, brain, immune cells) is of prognostic significance. Characterization of Brx will likely lead to the development of antibody-based screening tests for staging of malignant state in tissues such as breast, ovarian, endometrial, testicular, brain and immune malignancies. Accordingly, the methods of detecting the presence, absence, level of expression and tissue-distribution of Brx that are mentioned above and below are used to detect disease states mediated by the expression of Brx protein.

In regards to estrogen-responsive tissue, only a few proteins bind to estrogen receptors and Brx appears to be one of these. In particular, detection of Brx using these methods may permit further delineation and analysis of ER function in tumor progression and treatment and perhaps permit identification of a role for Brx or its derivatives in tamoxifen-resistant tumors.

5. Drug Screening Assays

The methods of detecting the presence, absence, level of expression, and tissue-distribution of Brx that are mentioned above and below are used to detect molecules and administration regimes that interact with and/or alter the pattern of expression of Brx and give rise to defined phenotypes. For example, where the effect of a putative drug on Brx expression is to be determined, the drug will be administered to an organism, tissue, or cell that expresses or is engineered to express Brx. Expression levels, or expression products will be assayed as described herein and the results compared with results from organisms, tissues, or cells similarly treated, but without the drug being tested.

6. Therapeutic Applications

a. Cancer Treatment

(1) Selection of Patients

The Brx and Brx fragments detected by the methods of the invention are used to treat cancer patients. The claimed methods are effective against a range of different cancer types, including not limited to tumors of immune and reproductive tissues, e.g., germinal epithelium of both ovary and testis, mammary epithelium, and epithelial and stromal cells of the endometrium, lymphoproliferative disorders such as chronic myelogenous leukemia, and Burkitt's lymphoma.

(2) Pre-Testing for Efficacy Against a Particular Cancer

In order to assess how well the methods of the invention may be expected to work, the clinician can pre-test the efficacy of the treatment of a particular tumor type either in vitro or in vivo.

For in vitro tests, cells derived from the tumor are grown in tissue culture. The growth inhibiting effect may be assessed using a number of commonly used assays, such as cell counts, or radioactive thymidine incorporation, or a methylcellulose assay (Lunardi-Iskandar et al., Clin. Exp. Immunol., 60:285-293 (1985)).

Both in vivo and in vitro testing may be accomplished using techniques well known to persons of ordinary skill in the art, based upon the guidance provided herein.

(3) Methods of Administration and Dosages

Administration of the above antibody preparations to a cancer patient can be achieved in various ways known to skilled practitioners. The above antibody preparations can be injected intratumorly: the tumor, the placement of the needle and release of the contents of the syringe may be visualized either by direct observation (for easily accessible tumors such as surface tumors or tumors easily exposed by surgical techniques), by endoscopic visualization, or by electromagnetic imaging techniques such as ultrasound, magnetic resonance imaging (MRI), CT scans. The antibody preparations can also be administered via injection into the bloodstream using a cannula or catheter; the vein or artery is selected to maximize delivery of cells to the tumor or affected tissue. In cystic or vesicular tumors or tissues, the compounds may be delivered intracystically or intravesicularly.

It is contemplated that the antibody preparations will be administered under the guidance of a physician. The concentration to be administered at a given time and to a given patient will vary from 0.1 mg-100 mg and preferably 0.1-10 mg per day per patient. Generally, the dosage to be administered is the amount necessary to reduce cancer cell growth and/or to destroy cancer cells and/or preferably to eradicate the cancer. The exact number is a function of the size and compactness (or diffuseness) of the particular transformed cell mass to be treated, and the distance or accessibility of the tissue to be treated from the point of administration of the cells. More than one administration may be necessary. As with any medical treatment, the supervising physician will monitor the progress of the treatment, and will determine whether a given administration is successful and sufficient, or whether subsequent administrations are needed.

According to an implementation, the present invention provides a kit comprising: (a) a polypeptide or plurality of polypeptides selected from the group consisting of (i) polypeptides that specifically bind to a ΔN3-Brx protein at about the C-terminal region, said C-terminal region comprising SEQ ID NO:2, (ii) polypeptides that specifically bind to a full length Brx protein (SEQ ID NO:1) at about the C-terminal region, said C-terminal region comprising SEQ ID NO:2; (iii) polypeptides that specifically bind at about the C-terminal region of a Brx protein derived from the same gene as the ΔN3-Brx protein, said Brx protein(s) being larger than the ΔN3-Brx protein and including but not limited to a protein comprising SEQ ID NO:1, (iv) polypeptides that specifically binds to a protein comprising SEQ ID NO. 2, and (v) combinations thereof; and (b) a reagent for detecting a monoclonal antibody-ΔN3-Brx protein complex in a patient or in a sample of tissue from the patient.

The reagent used may be any suitable reagent as would be known to persons of ordinary skill in the art. Preferably, the reagent is a label. More preferably, the label is a free label. Even more preferably, the label is bound to a solid support.

The kit may further comprise a container for containing the monoclonal antibody. Further, the kit may comprise instructions for the use of the kit. The kit may be a diagnostic kit, an immunization kit or a therapeutic kit. For example, the kit may be used to diagnose, prevent or treat a condition in a patient. The condition may be a disease of reproductive tissue, a disease of immune tissue or a combination thereof. The condition may be a disease of the epithelium, immune cells, ovary, breast, endometrium, spleen, brain or combination thereof. The condition may be a malignant tumor. The condition may be cancer. The condition may be breast cancer, ovarian cancer, endometrial cancer, testicular cancer, brain malignancy, immune malignancy, or combination thereof.

According to an implementation of the present invention, a method is provided for detecting ΔN3-Brx protein. The composition may be introduced to the tissue in vitro or in vivo.

According to another implementation of the present invention, a method is provided for protecting a patient against infection and/or colonization by ΔN3-Brx protein comprising: administering to the patient an effective amount of a composition comprising a polypeptide or plurality of polypeptides selected from the group consisting of (i) polypeptides that specifically bind to a ΔN3-Brx protein at about the C-terminal region, said C-terminal region comprising SEQ ID NO:2; (ii) polypeptides that specifically bind to a full length Brx protein (SEQ ID NO:1) at about the C-terminal region, said C-terminal region comprising SEQ ID NO:2; (iii) polypeptides that specifically bind at about the C-terminal region of a Brx protein derived from the same gene as the ΔN3-Brx protein, said Brx protein(s) being larger than the ΔN3-Brx protein and including but not limited to a protein comprising SEQ ID NO:1, (iv) polypeptides that specifically binds to a protein comprising SEQ ID NO. 2, and (v) combinations thereof.

The composition may be administered orally, parenterally, enterically, gastrically, topically, subcutaneously, locally, systematically or combinations thereof. The composition may be administered in a dosage of about 0. 1 mg to about 1000 mg per patient per twenty-four hour period of time. Preferably, the composition is administered in a dosage of about 0.1 mg to about 100 mg per patient per twenty-four hour period of time, and more preferably, the composition is administered in a dosage of about 0.1 mg to about 10 mg per patient per twenty-four hour period of time. Further, the composition may be administered once or multiple times during a twenty-four hour period of time.

According to a further implementation of the present invention, a method is provided for producing a monoclonal antibody comprising: introducing into the cells of an animal an immunogen comprising the polypeptide of SEQ ID NO:2; and recovering the monoclonal antibody from the cells of the animal. The monoclonal antibody may comprise a polypeptide or plurality of polypeptides selected from the group consisting of (i) polypeptides that specifically bind to a ΔN3-Brx protein at about the C-terminal region, said C-terminal region comprising SEQ ID NO:2, (ii) polypeptides that specifically bind to a full length Brx protein (SEQ ID NO:1) at about the C-terminal region, said C-terminal region comprising SEQ ID NO:2; (iii) polypeptides that specifically bind at about the C-terminal region of a Brx protein derived from the same gene as the ΔN3-Brx protein, said Brx protein(s) being larger than the ΔN3-Brx protein and including but not limited to a protein comprising SEQ ID NO:1, (iv) (iv) polypeptides that specifically binds to a protein comprising SEQ ID NO. 2, and (v) combinations thereof.

(4) Monitoring Treatment

Tumor regression and other parameters of successful treatment are assessed by methods known to persons of skill in the art. This includes any imaging techniques that are capable of visualizing cancerous tissues (e.g., MRI), biopsies, methods for assessing metabolites produced by the cancer tissue or affected tissue in question, the subjective wellbeing of the patient, etc.

7. Polynucleotides and Cell Lines

Implementations of the present invention further provide polynucleotides and cell lines. In an implementation of the present invention, a polynucleotide is provided comprising: (i) a nucleic acid sequence that encodes a polypeptide that specifically binds to a ΔN3-Brx protein at about the C-terminal region, said C-terminal region comprising SEQ ID NO:2; (ii) a nucleic acid sequence that encodes a polypeptide that specifically binds to a full length Brx protein (SEQ ID NO:1) at about the C-terminal region, said C-terminal region comprising SEQ ID NO:2; (iii) a nucleic acid sequence that encodes a polypeptide that specifically binds at about the C-terminal region of a Brx protein derived from the same gene as the ΔN3-Brx protein, said Brx protein(s) being larger than the ΔN3-Brx protein and including but not limited to a protein comprising SEQ ID NO:1, (iv) a nucleic acid sequence that encodes a polypeptide that specifically binds to a protein comprising SEQ ID NO. 2, or (v) combinations thereof. The polynucleotides of the present invention may be used in vectors and the like, for example, without limitation.

In another implementation, the present invention provides a transformed cell line comprising: (i) a recombinant cell expressing a polypeptide that specifically binds to a ΔN3Brx protein at about the C-terminal region, said C-terminal region comprising SEQ ID NO:2; (ii) a recombinant cell expressing a polypeptide that specifically binds to a full length Brx protein (SEQ ID NO:1) at about the C-terminal region, said C-terminal region comprising SEQ ID NO:2; or (iii) a recombinant cell expressing a polypeptide that specifically binds at about the C-terminal region of a Brx protein derived from the same gene as the ΔN3-Brx protein, said Brx protein(s) being larger than the ΔN3-Brx protein and including but not limited to a protein comprising SEQ ID NO: 1, (iv) a recombinant cell expressing a polypeptide that specifically binds to a protein comprising SEQ ID NO. 2, (v) or combinations thereof. The polypeptide may be a monoclonal antibody. The recombinant cell may be a hybridoma. The recombinant cell may be a trioma.

Based upon the guidance provided herein with regard to the polypeptides of the present invention, the preparation and use of polynucleotides and cell lines would be well within the skill of the art.

The present invention has been described in connection with the preferred embodiments. These embodiments, however, are merely for example and the invention is not restricted thereto. Any examples described herein are illustrative of preferred embodiments of the inventive subject matter and are not to be construed as limiting the inventive subject matter thereto. It will be understood by persons skilled in the art that other variations and modifications can easily be made within the scope of the invention as defined by the appended claims.

What is claimed is:

1. A monoclonal antibody comprising:
(i) a polypeptide that specifically binds to a ΔN3-Brx protein at about the C-terminal region, said C-terminal region comprising SEQ ID NO:2;
(ii) a polypeptide that specifically binds to a full-length Brx protein at about the C-terminal region, said C-terminal region comprising SEQ ID NO:2;
(iii) a polypeptide that specifically binds at about the C-terminal region of a Brx protein derived from the same gene as the ΔN3-Brx protein, said Brx protein(s) being larger than the ΔN3-Brx protein and including but not limited to a protein comprising SEQ ID NO:1; or
(iv) a polypeptide that specifically binds to a protein comprising SEQ ID NO. 2.

2. The monoclonal antibody of claim 1, wherein the polypeptide specifically binds at about amino acids 960 to 1428 of SEQ ID NO:1.

3. The monoclonal antibody of claim 1, wherein the polypeptide specifically binds at about amino acids 1 to 1428 of SEQ ID NO:1.

4. The monoclonal antibody of claim 1, wherein the C-terminal region has the ability to specifically bind to GST-RXRβ, ERα/β and other NHRs, the ability to modestly enhance activation by the estrogen receptor, and the ability to specifically bind to transcription factors known to be involved in mitogenesis: c-Jun, c-Fos and ATF-2.

5. The monoclonal antibody of claim 1, wherein the monoclonal antibody is mouse monoclonal antibody.

6. The monoclonal antibody of claim 1, wherein the method is used to treat, diagnose or prevent a condition of a patient.

7. The monoclonal antibody of claim 6, wherein the condition is cancer.

8. A monoclonal antibody prepared by a process comprising:
introducing into an animal a ΔN3-Brx protein comprising SEQ ID NO:2; and
recovering a monoclonal antibody from the animal.

9. The monoclonal antibody of claim 8, wherein the monoclonal antibody comprises:
(i) a polypeptide that specifically binds to a ΔN3-Brx protein at about the C-terminal region, said C-terminal region comprising SEQ ID NO: 1;
(ii) a polypeptide that specifically binds to a full-length Brx protein at about the C-terminal region, said C-terminal region comprising SEQ ID NO: 1;
(iii) a polypeptide that specifically binds at about the C-terminal region of a Brx protein derived from the same gene as the ΔN3-Brx protein, said Brx protein being larger than the ΔN3-Brx protein and comprising SEQ ID NO:1; or
(iv) a polypeptide that specifically binds to a protein comprising SEQ ID NO. 2.

10. The polypeptide of claim 8, wherein the animal is a mouse.

11. A composition comprising:
(a) a polypeptide or plurality of polypeptides selected from the group consisting of (i) polypeptides that specifically bind to a ΔN3-Brx protein at about the C-terminal region, said C-terminal region comprising SEQ ID NO:1; (ii) polypeptides that specifically bind to a full length Brx protein at about the C-terminal region, said C-terminal region comprising SEQ ID NO:1; (iii) polypeptides that specifically bind at about the C-terminal region of a Brx protein derived from the same gene as the ΔN3-Brx protein, said Brx protein being larger than the ΔN3-Brx protein and comprising SEQ ID NO:1; (iv) a polypeptide that specifically binds to a protein comprising SEQ ID NO. 2; and (v) combinations thereof; and
(b) a pharmaceutically acceptable carrier.

12. The composition of claim 11, wherein the composition is a pharmaceutical composition.

13. The composition of claim 12, wherein the pharmaceutical composition is effective in treating or diagnosing a condition in a patient.

14. The composition of claim 13, wherein the condition is a disease of reproductive tissue, a disease of immune tissue or a combination thereof.

15. The composition of claim 13, wherein the condition is a disease of the epithelium, immune cells, ovary, breast, endometrium, spleen, brain or combination thereof.

16. The composition of claim 13, wherein the condition is cancer or a malignant tumor.

17. The composition of claim 13, wherein the condition is breast cancer, ovarian cancer, endometrial cancer, testicular cancer, brain malignancy, immune malignancy, or combination thereof.

18. The composition of claim 11, wherein the pharmaceutically acceptable carrier is selected from the group consisting of mannitol, lactose, starch, magnesium stearate, sodium saccharin, talcum cellulose, glucose, magnesium, carbonate, sodium acetate, sodium chloride, potassium chloride, calcium chloride, sodium lactate and combinations thereof.

19. The composition of claim 11, wherein the composition is in the form of a solution, suspension, tablet, pill, capsule, microcapsule, powder, particle, gel, cream, lotion, ointment, aerosol, or combination thereof.

20. The composition of claim 11, said composition being prepared by a process comprising:
introducing a ΔN3-Brx protein or fragment thereof into cells of an animal; said ΔN320 Brx protein comprising SEQ ID NO: 2; and
recovering the monoclonal antibody from the cells of the animal.

21. A polynucleotide comprising:
(i) a nucleic acid sequence that encodes a polypeptide that specifically binds to a ΔN3-Brx protein at about the C-terminal region, said C-terminal region comprising SEQ ID NO:2;
(ii) a nucleic acid sequence that encodes a polypeptide that specifically binds to a full length Brx protein (SEQ ID NO: 1) at about the C-terminal region, said C-terminal region comprising SEQ ID NO:2;
(iii) a nucleic acid sequence that encodes a polypeptide that specifically binds at about the C-terminal region of any Brx protein derived from the same gene as the ΔN3-Brx protein, said Brx protein being larger than the ΔN3-Brx protein and including but not limited to a protein comprising SEQ ID NO:1; or
(iv) a nucleic acid sequence that encodes a polypeptide that specifically binds to a protein comprising SEQ ID NO. 2.

22. A transformed cell line comprising:
(i) a recombinant cell expressing a polypeptide that specifically binds to a ΔN3Brx protein at about the C-terminal region, said C-terminal region comprising SEQ ID NO:2;
(ii) a recombinant cell expressing a polypeptide that specifically binds to a full length Brx protein (SEQ ID NO:1) at about the C-terminal region, said C-terminal region comprising SEQ ID NO:2;
(iii) a recombinant cell expressing a polypeptide that specifically binds at about the C-terminal region of any Brx protein derived from the same gene as the ΔN3-Brx protein, said Brx protein(s) being larger than the ΔN3-Brx protein and including but not limited to a protein comprising SEQ ID NO:1; or
(iv) a recombinant cell expressing a polypeptide that specifically binds to a protein comprising SEQ ID NO. 2.

23. A method for detecting ΔN3-Brx protein in a patient comprising:
introducing to the tissue of a patient a composition comprising a polypeptide or plurality of polypeptides selected from the group consisting of (i) polypeptides that specifically bind to a ΔN3-Brx protein at about the C-terminal region, said C-terminal region comprising SEQ ID NO:2, (ii) polypeptides that specifically bind to a full length Brx protein (SEQ ID NO:1) at about the C-terminal region, said C-terminal region comprising SEQ ID NO:2, (iii) polypeptides that specifically bind at about the C-terminal region of a Brx protein derived from the same gene as the ΔN3-Brx protein, said Brx protein being larger than the ΔN3-Brx protein and including but not limited to a protein comprising SEQ ID NO:1, (iv) a polypeptide that specifically binds to a protein comprising SEQ ID NO. 2, and (v) a combination thereof; and
detecting the presence, absence and/or level of a brx gene protein product in the tissue.
